(19) United States
(12) Patent Application Publication
(10) Pub. No.: US 2021/0230545 A1
(43) Pub. Date: Jul. 29, 2021

(54) USE OF IL-12 TO ALTER EPIGENETIC EFFECTOR PROGRAMS IN CD8 T CELLS

(71) Applicant: St. Jude Children's Research Hospital, Memphis, TN (US)

(72) Inventors: Caitlin Zebley, Memphis, TN (US); Hossam Abdelsamed, Pittsburgh, PA (US); Benjamin Youngblood, Memphis, TN (US)

(73) Assignee: St. Jude Children's Research Hospital, Memphis, TN (US)

(21) Appl. No.: 17/258,533

(22) PCT Filed: Jul. 8, 2019
(86) PCT No.: PCT/IB2019/055801

§ 371 Date: Jan. 7, 2021

Related U.S. Application Data
(60) Provisional application No. 62/695,298, filed on Jul. 9, 2018.

(51) Int. Cl. C12N 5/0783; C07K 14/725; C12Q 1/6876; A61K 35/17

(52) U.S. Cl. CPC C12N 5/0636; C07K 14/7051; C12Q 1/6876; A61K 35/17; C12N 2501/515; C12N 2501/51; C12N 2501/2312; C12Q 2600/154; C12N 2501/998

(57) ABSTRACT

Provided herein are methods and compositions for modulating T-cell activity by incubating a CD8 T cell with a signal 3 cytokine, such as IL-12. Incubation of naïve CD8 T cells, particularly, with a signal 3 cytokine can acquire long-lived memory associated gene expression characteristic of the stem cell memory subset of CD8 T cells. Further, incubation with signal 3 cytokines can induce changes to the epigenetic profile of naïve CD8 T cells that are more characteristic of bona fide T_{scm} cells than in vitro generated cells using traditional differentiation protocols. On account of epigenetic profiles being preserved during in vivo homeostasis, signal 3 cytokines such as IL-12 can be used to engineer a T cell population with the desired epigenetic profile that maintains effector functions and proliferative capacity.

### CROSS-REFERENCE TO RELATED APPLICATIONS

This application is a national stage filing under 35 U.S.C. 371 of PCT/IB2019/05801 filed Jul. 8, 2019, which International Application was published by the International Bureau in English on Jan. 16, 2020, and application claims priority from U.S. Provisional Patent Application No. 62/695,298, filed Jul. 9, 2018, which applications are hereby incorporated in their entirety by reference in this application.

### STATEMENT OF FEDERALLY SPONSORED RESEARCH OR DEVELOPMENT

This invention was made with government support under grant number AI114442 awarded by the National Institutes of Health. The government has certain rights in the invention.

### FIELD OF THE INVENTION

The invention relates to the field of cell biology and immunology. In particular, the invention relates to a method for modulating T-cell activity by incubating T cells with signal 3 cytokines, including IL-12. Exposure to signal 3 cytokines can establish phenotypic and epigenetic profiles to maintain effector function and proliferative capacity. The methods and compositions can be used to treat symptoms of chronic infections and cancer.

### BACKGROUND OF THE INVENTION

Following an infection, naïve CD8 T cells are stimulated by dendritic cells (DC) displaying pathogen-derived peptides on MHC class I molecules (signal 1) and costimulatory molecules (signal 2). Additionally, pathogen-induced inflammatory cytokines also act directly on the responding CD8 T cells to regulate their expansion and differentiation. In particular, both type I interferons (IFNs) and IL-12 have been described as critical survival signals (signal 3) for optimal CD8 T cell accumulation during the expansion phase. Furthermore, expansion in numbers of antigen-specific CD8 T cells is coupled with their acquisition of effector functions to combat the infection.

Chimeric antigen receptor (CAR) T cell therapy is revolutionizing the field of cancer immunotherapy. Although most current CAR T cell protocols use the total pool of T cells, the CD8 T-cell subset of stem cell memory (T_{scm}) cells have an enhanced ability to eradicate tumor and proliferate. Given these properties, T_{scm} have been considered an ideal CD8 T-cell subset for adoptive cell transfer. However, T_{scm} comprise a small percentage of the existing T cell population. Current protocols strive to generate T_{scm} from the abundant naïve CD8 T cells. These protocols have largely overlooked the molecular mechanisms that govern CD8 T-cell differentiation.

Previous reports documents the DNA methylation profile of human naïve and memory CD8 T cell subsets, including T_{scm}, that are associated with the respective state of differentiation, and these signatures have been used to track the differentiation status of in vitro generated effector and memory T cells. While applying current protocol conditions induces phenotypic changes in naïve CD8 T cells, these changes are not reflected at the epigenetic level. Notably, the epigenetic profile of in vitro generated T_{scm} has been found to be different than that of a bona fide T_{scm}. The methods and compositions disclosed herein utilize the discovery presented herein that in order for human naïve CD8 T cells to acquire long-lived, memory-associated gene expression, they require co-stimulation with signal 3 cytokines. In fact, varying in vitro culture conditions with different cytokines induces changes in the phenotype of naïve human CD8 T cells and, importantly, these changes are reflected at the epigenetic level.

### SUMMARY OF THE INVENTION

Provided herein are methods and compositions for modulating T-cell activity by incubating a CD8 T cell with a signal 3 cytokine, such as IL-12. Incubation of naïve CD8 T cells, particularly, with a signal 3 cytokine can acquire long-lived memory associated gene expression characteristic of the stem cell memory subset of CD8 T cells. Further, incubation with signal 3 cytokines can induce changes to the epigenetic profile of naïve CD8 T cells that are more characteristic of bona fide T_{scm} cells than in vitro generated cells using traditional differentiation protocols. On account of epigenetic profiles being preserved during in vivo homeostasis, signal 3 cytokines such as IL-12 can be used to engineer a T cell population with the desired epigenetic profile that maintains effector functions and proliferative capacity. Accordingly, provided herein are populations of CD8 T cells having been incubated with a signal 3 cytokine that contain a higher percentage of cells exhibiting the epigenetic profile of T_{scm} cells than those populations produced with traditional protocols. In some embodiments, the CD8 T Cells are CAR T cells for the treatment or prevention of disease. Thus, the methods and compositions can be used to treat symptoms of chronic infections and cancer.

### BRIEF DESCRIPTION OF THE DRAWINGS

FIG. 1 presents a flow-cytometry strategy for isolating naïve CD8 T cells from healthy human donors. Naïve CD8 T cells were then cultured in vitro under varying conditions with subsequent phenotypic and epigenetic analysis.

FIG. 2 shows the IFNγ expression and corresponding epigenetic profile after culturing naïve CD8 T cells in culture with varying cytokines for one week.

FIG. 3 demonstrates IFNγ expression and corresponding methylation profile of CD8 T cells at the 7 day and 14 day time points of incubation with IL-12 and/or TCR.

FIG. 4 shows the phenotypic variation induced under one week of differing in vitro cell culture conditions.

FIG. 5 demonstrates a phenotypic analysis comparing CD45RO⁻ vs CD45RO⁺ cells.

FIG. 6 presents a bisulfite sequencing analysis for IFNγ promoter comparing cells cultured under varying in vitro conditions.

FIG. 7 shows Coinfection of C57BL/6 mice with LM and LCMV induces demethylation at the IFNγ downstream region in effector CD8 T cells.

FIG. 8 presents data following infection of C57BL/6 mice with LM and subsequently infected with chronic LCMV at D60. DNA methylation analysis is shown for memory CD8 T cells at the IFNγ downstream locus.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Compositions and methods are provided herein for modulating T-cell activity by exposing at least one CD8 T cell to a signal 3 cytokine in order to enhance effector functions and proliferative capacity of the CD8 T cell. Signal 3 inflammatory cytokines regulate multiple aspects of the CD8 T cell response. Co-stimulation with signal 3 cytokines has now been shown to help provide long-lived memory associated gene expression.

CD8 T cells undergo activation by interaction of the T-cell receptor (TCR) on the CD8 T cell with antigen bound to MHC-I on antigen presenting cells. Once activated the T cell undergoes clonal expansion to increase the number of cells specific for the target antigen. When exposed to infected or dysfunctional somatic cells having the specific antigen for which the TCR is specific, the activated CD8 T cells release cytokines and cytotoxins to eliminate the infected or dysfunctional cell. Thus, as used herein, the term “activation” or “stimulation” of a CD8 T cell refers to engagement of a T cell receptor (e.g., TCR or CAR) with an antigen. In some embodiments, CD8 T cells can be activated by anti-CD3 and/or anti-CD28 antibodies. In specific embodiments, CD8 T cells can be activated by antigens presented from tumor cells or self-antigens as described elsewhere herein.

The release of cytokines and cytotoxins by CD8 T cells in response to an antigen is referred to herein as “effector functions”. In some embodiments, the cytokines and cytotoxins released are specific for the activating antigen. Likewise, the term “effector potential” refers to the ability of CD8 T cells to activate effector functions upon activation. The term “T-cell activity” refers to any of the following: cytokine production (e.g., IFNγ and IL-2) upon activation; expression of cytotoxic molecules (e.g., granzyme B and perforin) upon activation; rapid cell division upon activation; cytolysis of antigen presenting cells; IL-7 and IL-15 mediated homeostatic proliferation; and in vivo trafficking to lymphoid tissues or sites of antigen presentation. Moreover, “T-cell activity” can refer to the persistence of immunological memory in the absence of antigen.

The methods and compositions disclosed herein utilize the previously unknown ability of signal 3 cytokines to enhance effector functions and proliferative capacity of the CD8 T cell. As used herein “signal 3 cytokines” refer to type I interferons (IFN, i.e., IFN-α, -β) and IL-12. These signal 3 cytokines have been described as critical survival signals for optimal CD8 T cell accumulation during the expansion phase. Expansion in numbers of antigen-specific CD8 T cells is coupled with their acquisition of effector functions to combat the infection. However, while traditional methods of expansion and differentiation of T cells may induce phenotypic changes among naïve CD8 T cells, these changes are not necessarily reflected at the epigenetic level. It has now been shown that signal 3 cytokines can induce changes at the epigenetic level and phenotypic changes both of which are consist with the ideal T_{scm} cell type. Thus, provided herein are methods for using signal 3 cytokines for generating memory cells from naïve CD8 T cells by inducing epigenetic changes that result in the desired T_{scm} epigenetic profile having enhanced effector functions and proliferative capacity.

Although current protocols for expansion and differentiation of T cells strive to produce T_{scm} cells, actual levels of T_{scm} cells remain relatively low. However, by utilizing signal 3 cytokines as described in the methods herein, a population of CD8 T cells can be produced having a greater ratio of T_{scm} cells to the total population than the same ratio using current methods. Further, exposure to signal 3 cytokines can provide the important epigenetic profile that characterizes T_{scm} cells having enhanced effector functions and proliferative capacity. The populations of T cells produced by the methods disclosed herein can be CAR T cells used for adoptive cell transfer and the treatment of disease. Accordingly, pharmaceutical compositions and methods are provided for treatment of diseases, such as cancer, comprising the population of cells produced by exposure to signal 3 cytokines.

### II. Methods of Modulating T-Cell Activity

Compositions and methods are provided herein for the modulating T-cell activity of CD8 T cells by exposing the CD8 T cell to signal 3 cytokines, such as IL-12. Modulating T-cell activity refers to increase or decreasing T-cell activity relative to an appropriate control. Such modulation, modulating, alteration, or altering includes enhancing or repressing effector functions, enhancing or repressing cytokine production (e.g., IFNγ and IL-2), enhancing or repressing expression of cytotoxic molecules (e.g., granzyme B and perforin), enhancing or repressing cell division, enhancing or repressing cytolysis of antigen presenting cells, enhancing or repressing proliferative capacity, enhancing or repressing IL-7 and IL-15 mediated homeostatic proliferation, enhancing or repressing in vivo trafficking to lymphoid tissues or sites of antigen presentation. Moreover, modulating T-cell activity can refer to the increase or decrease of immunological memory in the absence of antigen. In specific embodiments, modulating T-cell activity can refer to enhancing or increasing effector functions or proliferative capacity. In specific embodiments, the methylation status or methylation level of at least one genomic locus is decreased in order to increase T-cell activity by exposure of the CD8 T cells to signal 3 cytokines.

The term “methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively. By “hypermethylation” or “increased methylation” is meant an increase in methylation of a region of DNA (e.g., a genomic locus as disclosed herein) that is considered statistically significant over levels of a control population. “Hypermethylation” or “increased methylation” may refer to increased levels seen in a subject over time or can refer to the methylation level relative to the methylation status of the same locus in a naïve T cell.

Moreover, the activity of CD8 T cells can be predicted based on measuring the methylation status of one or more than one genomic locus. For example, in specific embodiments, the methylation profile of memory CD8 T cells produced by incubation with a signal 3 cytokine is different than the methylation profile of memory CD8 T cells produced in the absence of signal 3 cytokines. Accordingly, a “methylation profile” refers to a set of data representing the methylation states or levels of one or more loci within a molecule of DNA from e.g., the genome of an individual or cells or sample from an individual. The profile can indicate the methylation state of every base in an individual, can comprise information regarding a subset of the base pairs (e.g., methylation state of an effector locus, or region surrounding an effector locus) in a genome, or can comprise information regarding regional methylation density of each locus. In some embodiments, a methylation profile refers to the methylation states or levels of one or more genomic loci (e.g., effector loci or biomarkers) described herein. In more specific embodiments, a methylation profile refers to the methylation status of a transcription factor loci for Tcf7, Myc, T-bet, eomesodermin (Eomes), and/or Foxp1, at least one CpG site in the CCR7 and/or CD62L loci, a region located within 1 kb of the transcription start site of a nucleic acid sequence encoding IFNγ, granzyme K, GzmB, or Prf1, or any gene, promoter, transcription factor, 3′ untranslated region (UTR), or regulator of cellular proliferation.

The terms “methylation status” or “methylation level” refer to the presence, absence, and/or quantity of methylation at a particular nucleotide, or nucleotides within a portion of DNA. The methylation status of a particular DNA sequence (e.g., an effector locus, a DNA biomarker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value” or “methylation level.” A methylation value or level can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, represents the methylation status and can thus be used as a quantitative indicator of methylation status. This is of particular use when it is desirable to compare the methylation status of a sequence in a sample to a threshold value. A “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC).

The terms “measuring” and “determining” are used interchangeably throughout, and refer to methods which include obtaining a subject sample and/or detecting the methylation status or level of a biomarker(s) in a sample. In one embodiment, the terms refer to obtaining a subject sample and detecting the methylation status or level of one or more biomarkers in the sample. In another embodiment, the terms “measuring” and “determining” mean detecting the methylation status or level of one or more biomarkers in a subject sample. Measuring can be accomplished by methods known in the art and those further described herein including, but not limited to, quantitative polymerase chain reaction (PCR). The term “measuring” is also used interchangeably throughout with the term “detecting.”

The T-cell activity of a CD8 T cell can be modulated (e.g., increased) by contacting or incubating the CD8 T cell with a signal 3 cytokine. In specific embodiments, the T-cell activity of a CD8 T cell can be modulated by incubating a naïve CD8 T cell with a signal 3 cytokine, such as IL-12, and an antigen that activates the TCR or CAR of the naïve CD8 T cell. Such contacting and incubating can be performed in vivo, wherein the cell is in the body of a subject mammal; in vitro, wherein the cell is propagated in culture; or ex vivo, wherein the cell has been taken from a subject mammal and is preserved in culture. For example, a signal 3 cytokine such as IL-12 can be administered to a subject in order to achieve contact with a CD8 T cell or can be added to a cell culture medium comprising a CD8 T cell. Likewise, a signal 3 cytokine such as IL-12 can be administered along with an activating antigen to a subject in order to achieve contact with a CD8 T cell and activation or can be delivered without an activating antigen in order to rely on separate activation of the CD8 T cell by an endogenous or exogenous antigen. In specific embodiments, contacting a signal 3 cytokine with a CD8 T cell will enhance or increase effector functions and proliferative capacity. Exposure of a signal 3 cytokine to naïve CD8 T cells can decrease the methylation status of a particular genomic locus or methylation profile which, when activated, can increase T-cell activity by enhancing cytokine production (e.g., IFNγ and IL-2), enhancing expression of cytotoxic molecules (e.g., granzyme B and perforin), enhancing cell division, enhancing cytolysis of antigen presenting cells, enhancing IL-7 and IL-15 mediated homeostatic proliferation, enhancing in vivo trafficking to lymphoid tissues or sites of antigen presentation or increasing persistence of immunological memory in the absence of antigen.

The T-cell activity of any T cell can be modulated (e.g., increased) by contacting the cell with a signal 3 cytokine. For example the T-cell activity of any CD8 T cell (i.e., CD8+ T cell) can be increased, following activation, by contacting the cell with a signal 3 cytokine using the methods disclosed herein. In specific embodiments, T-cell activity is increased by incubating a CD8 T cell, such as a naïve CD8 T cell, with IL-12 and an activating antigen. Increase in T-cell activity (e.g., increasing effector functions and proliferative capacity) can refer to at least a 95% increase, at least a 90% increase, at least a 80% increase, at least a 70% increase, at least a 60% increase, at least a 50% increase, at least a 40% increase, at least a 30% increase, at least a 20% increase, at least a 10% increase, or at least a 5% increase of the cytokine production (e.g., IFNγ and/or IL-2), expression of cytotoxic molecules (e.g., granzyme B and/or perforin), cell division, cytolysis of antigen presenting cells, IL-7 and IL-15 mediated homeostatic proliferation, in vivo trafficking to lymphoid tissues or sites of antigen presentation or increasing persistence of immunological memory in the absence of antigen when compared to an appropriate control, such as a naïve T cell, an unmodified T cell, or a T cell that has not been exposed to a signal 3 cytokine.

In particular embodiments, the CD8 T cell is a T cell having a modified T-cell receptor, such as a CAR T cell. As used herein, a “chimeric antigen receptor” or “CAR” refers to an engineered receptor that grafts specificity for an antigen onto an immune effector cell (e.g., a human T cell). A chimeric antigen receptor typically comprises an extracellular ligand-binding domain or moiety and an intracellular domain that comprises one or more stimulatory domains. In some embodiments, the extracellular ligand-binding domain or moiety can be in the form of single-chain variable fragments (scFvs) derived from a monoclonal antibody, which provide specificity for a particular epitope or antigen (e.g., an epitope or antigen preferentially present on the surface of a cancer cell or other disease-causing cell or particle). The extracellular ligand-binding domain can be specific for any antigen or epitope of interest. In some embodiments, CD8 T cell is exposed to the signal 3 cytokine (e.g., IL-12) prior to the addition of the CAR. In other embodiments, a CD8 T cell having a CAR can be contacted with a signal 3 cytokine (e.g., IL-12) in order to increase effector functions and/or proliferative capacity.

According to the methods disclosed herein CD8 T cells can be incubated or contacted with a signal 3 cytokine in order to increase effector function and/or proliferative capacity and/or to establish a T_{scm} epigenetic profile. In some embodiments, the conditions for the incubation, stimulation, or activation of CD8 T cells include conditions whereby T cells of the culture-initiating composition proliferate or expand. For example, in some aspects, the incubation is carried out in the presence of an agent capable of activating one or more intracellular signaling domains of one or more components of a TCR complex, herein referred to as an activating antigen, stimulating antigen, activating agent, or stimulating agent, such as a CD3 zeta chain, or capable of activating signaling through such a complex or component. In some aspects, the incubation is carried out in the presence of an anti-CD3 antibody, and anti-CD28 antibody, anti-4-1BB antibody, for example, such antibodies coupled to or present on the surface of a solid support, such as a bead, and/or a cytokine, such as IL-2, IL-15, IL-7, and/or IL-21.

In specific embodiments naïve CD8 T cells can be incubated or contacted with a signal 3 cytokine in any condition suitable for the growth and expansion of the CD8 T cells. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to proliferate or activate the cells. In one example, the stimulating conditions include one or more agent, e.g., ligand, which turns on or initiates TCR/CD3 intracellular signaling cascade in a T cell, herein referred to a stimulating factors or stimulating antigens. Such agents can include antibodies, such as those specific for a TCR component and/or costimulatory receptor, e.g., anti-CD3, anti-CD28, anti-4-1BB, for example, bound to solid support such as a bead, and/or one or more cytokines. In some embodiments, the expansion method may further comprise the step of adding anti-CD3 and/or anti CD28 antibody to the culture medium (e.g., at a concentration of at least about 0.5 ng/ml). The expansion method may further comprise the step of adding IL-2 and/or IL-15 and/or IL-7 and/or IL-21 to the culture medium (e.g., wherein the concentration of IL-2 is at least about 10 units/ml). In particular embodiments, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

The incubation or contacting time and temperature can be any time and temperature suitable for the specific cells in use. For example, the time of incubation can be from 6 hours to 180 days, 12 hours to 60 days, 1 day to 30 days, 4 days to 20 days, 6 days to 18 days, or 7 days to 14 days. The large range of incubation time is first of all due to the fact that samples from different donors may behave very differently. Also it was shown that the lymphocytes from different body samples have very different growth rates. In specific embodiments, the naïve CD8 T cells can be incubated with a signal 3 cytokine and stimulating factor at 37° C. for 5-14 days, including 5, 6, 7, 8, 9, 10, 11, 12, 13, and 14 days.

In some embodiments a population of naïve T cells is contacted with a signal 3 cytokine, wherein the population contains a mix of CD8 T cells having CARs and CD8 T cells with the endogenous TCR. In some embodiments, the population of CD8 T cells that is contacted with a signal 3 cytokine includes at least about 30%, 40%, 45%, 50%, 55%, 60%, 65%, 70%, 80%, 90%, 95% or more, or 30-80%, 35-60%, 40-60%, 50-70%, or 60-80% CD8 T cells having a CAR. The methods and compositions disclosed herein can be used to increase the relative percentage of CD8 T cells that exhibit the T_{scm} phenotype and epigenetic profile following contact with an activating antigen. In specific embodiments, the population of CD8 T cells has at least 30%, 40%, 45%, 50%, 55%, 60%, 65%, 70%, 80%, 90%, 95% or more, or 30-80%, 35-60%, 40-60%, 50-70%, or 60-80% CD8 T cells that exhibit the T_{scm} phenotype and epigenetic profile following contact with a signal 3 cytokine (e.g., IL-12) and an activating antigen.

T-cell adoptive immunotherapy is a promising approach for cancer treatment. This strategy utilizes isolated human T cells that have been genetically-modified to enhance their specificity for a specific tumor-associated antigen. Genetic modification may involve the expression of a chimeric antigen receptor or an exogenous T cell receptor to graft antigen specificity onto the T cell. By contrast to exogenous T cell receptors, chimeric antigen receptors derive their specificity from the variable domains of a monoclonal antibody. Thus, CAR T cells induce tumor immunoreactivity in a major histocompatibility complex non-restricted manner. To date, T cell adoptive immunotherapy has been utilized as a clinical therapy for a number of cancers, including B cell malignancies (e.g., acute lymphoblastic leukemia (ALL), B cell non-Hodgkin lymphoma (NHL), and chronic lymphocytic leukemia), multiple myeloma, neuroblastoma, glioblastoma, advanced gliomas, ovarian cancer, mesothelioma, melanoma, and pancreatic cancer, among others. In some embodiments, CAR T cells having modulated methylation profiles are administered along with ICB therapy.

In specific embodiments, CAR-CD8 T cells having been contacted with a signal 3 cytokine may be adoptively transferred into a patient. Adoptive transfer T-cell therapy of CAR-CD8 T cells following contact with a signal 3 cytokine may also be used in combination with immune checkpoint inhibitors such as antibodies to PD-1/PD-L1 and/or CD80/CTLA4 blockade, small molecule checkpoint inhibitors, interleukins, e.g., IL-2 (aldesleukin).

In some embodiments, T-cell activity is increased in a patient having a chronic infection or cancer. In certain embodiments, the chronic infection is a chronic viral infection. For example, T-cell activity can be increased using the methods disclosed herein in a subject infected with influenza A virus including subtype H1N1, influenza B virus, influenza C virus, rotavirus A, rotavirus B, rotavirus C, rotavirus D, rotavirus E, SARS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E, or human immunodeficiency virus (HIV). In particular embodiment, the chronic viral infection is HIV, HCV, and/or herpes virus.

As used herein a “proliferative disease” or “cancer” includes, a disease, condition, trait, genotype or phenotype characterized by unregulated cell growth or replication as is known in the art; including leukemias, for example, acute myelogenous leukemia (AML), chronic myelogenous leukemia (CIVIL), acute lymphocytic leukemia (ALL), and chronic lymphocytic leukemia, AIDS related cancers such as Kaposi's sarcoma; breast cancers; bone cancers such as osteosarcoma, chondrosarcomas, Ewing's sarcoma, fibrosarcomas, giant cell tumors, adamantinomas, and chordomas; brain cancers such as meningiomas, glioblastomas, lower-grade astrocytomas, oligodendrocytomas, pituitary tumors, schwannomas, and metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of disease related gene expression in a cell or tissue, alone or in combination with other therapies.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

In particular embodiments, a signal 3 cytokine can be contacted with a CD8 T cell along with an immune modulating agent. As used herein, an “immune modulating agent” is an agent capable of altering the immune response of a subject. In certain embodiments, “immune modulating agents” include adjuvants (substances that enhance the body's immune response to an antigen), vaccines (e.g., cancer vaccines), and those agents capable of altering the function of immune checkpoints, including the CTLA-4, LAG-3, B7-H3, B7-H4, Tim3, BTLA, KIR, A2aR, CD200 and/or PD-1 pathways. Exemplary immune checkpoint modulating agents include anti-CTLA-4 antibody (e.g., ipilimumab), anti-LAG-3 antibody, anti-B7-H3 antibody, anti-B7-H4 antibody, anti-Tim3 antibody, anti-BTLA antibody, anti-KIR antibody, anti-A2aR antibody, anti CD200 antibody, anti-PD-1 antibody, anti-PD-L1 antibody, anti-CD28 antibody, anti-CD80 or -CD86 antibody, anti-B7RP1 antibody, anti-B7-H3 antibody, anti-HVEM antibody, anti-CD137 or -CD137L antibody, anti-OX40 or -OX40L antibody, anti-CD40 or -CD40L antibody, anti-GALS antibody, anti-IL-10 antibody and A2aR drug. For certain such immune pathway gene products, the use of either antagonists or agonists of such gene products is contemplated, as are small molecule modulators of such gene products. In certain embodiments, the “immune modulatory agent” is an anti-PD-1 or anti-PD-L1 antibody.

Thus, CD8 T cells contacted with a signal 3 cytokine to enhance effector response and proliferative capacity can be combined with blockade of specific immune checkpoints such as the PD-1 pathway. In specific embodiments, the CD8 T cells exhibit a T_{scm} epigenetic marker or epigenetic profile following contact with a signal 3 cytokine. These two therapies need not be given concurrently, but could also be given sequentially, beginning with epigenetic modulation and followed by checkpoint blockade. This is because epigenetic modulation induced alterations in gene expression pattern continue after cessation of treatment of tumor cells (Tsai et al. Cancer Cell 2012, 21: 430-446). As used herein, the term “immune checkpoints” means a group of molecules on the cell surface of CD4+ and CD8+ T cells. These molecules fine-tune immune responses by down-modulating or inhibiting an anti-tumor immune response. Immune checkpoint proteins are well known in the art and include, without limitation, PD-L1, as well as CTLA-4, PD-1, VISTA, B7-H2, B7-H3, B7-H4, B7-H6, 2B4, ICOS, HVEM, PD-L2, CD160, gp49B, PIR-B, KIR, TIM-3, LAG-3, HHLA2, butyrophilins, and BTLA (see, for example, WO 2012/177624). As used herein, “immune checkpoint blockade,” “ICB,” or “checkpoint blockade” refers to the administration of an agent that interferes with the production or activity of immune checkpoint proteins.

In certain embodiments, modified CD8 T cells having been exposed to a signal 3 cytokine to produce a T_{scm} phenotype and/or epigenetic profile upon activation as disclosed herein may be used in adoptive T cell therapies to enhance immune responses against cancer. For example, this disclosure relates to methods of treating cancer comprising a) collecting immune cells or CD8 T cells from a subject diagnosed with cancer; b) contacting the CD8 T cell with a signal 3 cytokine (e.g., IL-12); c) administering or implanting an effective amount of the immune cells or CD8 T cells following contact with the signal 3 cytokine into the subject diagnosed with cancer. In specific embodiments, the signal 3 cytokine is IL-12 and the CD8 T cell exhibits an epigenetic marker or epigenetic profile of a T_{scm} cell following activation.

In some embodiments the CD8 T cells are modified before or after contact with a signal 3 cytokine to express a chimeric antigen receptor (CAR) specific to a tumor associated antigen or neoantigen. In certain embodiments, the tumor associated antigen is selected from CD5, CD19, CD20, CD30, CD33, CD47, CD52, CD152(CTLA-4), CD274(PD-L1), CD340(ErbB-2), GD2, TPBG, CA-125, CEA, MAGEA1, MAGEA3, MART1, GP100, MUC1, WT1, TAG-72, HPVE6, HPVE7, BING-4, SAP-1, immature laminin receptor, vascular endothelial growth factor (VEGFA) or epidermal growth factor receptor (ErbB-1). In certain embodiments, the tumor associated antigen is selected from CD20, CD20, CD30, CD33, CD52, EpCAM, epithelial cells adhesion molecule, gpA33, glycoprotein A33, Mucins, TAG-72, tumor-associated glycoprotein 72, Folate-binding protein, VEGF, vascular endothelial growth factor, integrin αVβ3, integrin α5β1, FAP, fibroblast activation protein, CEA, carcinoembryonic antigen, tenascin, Ley , Lewis Y antigen, CAIX, carbonic anhydrase IX, epidermal growth factor receptor (EGFR; also known as ERBB1), ERBB2 (also known as HER2), ERBB3, MET (also known as HGFR), insulin-like growth factor 1 receptor (IGF1R), ephrin receptor A3 (EPHA3), tumor necrosis factor (TNF)-related apoptosis-inducing ligand receptor 1 (TRAILR1; also known as TNFRSF10A), TRAILR2 (also known as TNFRSF10B) and receptor activator of nuclear factor-KB ligand (RANKL; also known as TNFSF11) and fragments thereof.

In certain embodiments, the T-cells specific to a tumor antigen can be removed from a tumor sample (TILs) or filtered from blood. Subsequent activation and culturing is performed outside the body (ex vivo) and then they are transfused into the patient. Activation may be accomplished by exposing the T cells to tumor antigens.

### III. Methods for Selecting a Subset of CD8 T Cells

Methods and compositions are provided herein for selecting a population of CD8 T cells following incubation of the population of CD8 T cells with a signal 3 cytokine, and optionally activation, based on the methylation status of a specific locus or combination of loci or the methylation profile of a genomic region or complete genome of a CD8 T cell following activation. Selection of a subset of CD8 T cells with a desired activity can be performed by measuring the methylation status of a specific locus or combination of loci or the methylation profile of a genomic region or complete genome of a sample of CD8 T cells in order to predict the T cell activity of the population from which the sample was taken. In specific embodiments, CD8 T cells are selected after incubation with a signal 3 cytokine and activating antigen when the CD8 T cells exhibit a methylation marker or methylation profile of a Tsai, cell (i.e., T_{scm} marker or T_{scm} epigenetic profile). In specific embodiments, a population of CD8 T cells is selected when the population has at least 30%, 40%, 45%, 50%, 55%, 60%, 65%, 70%, 80%, 90%, 95% or more, or 30-80%, 35-60%, 40-60%, 50-70%, or 60-80% CD8 T cells that exhibit the T_{scm} phenotype and/or epigenetic profile following contact with a signal 3 cytokine (e.g., IL-12) and activation antigen.

The methylation status of any individual locus or a group of loci, such as effector loci, in the genome of a CD8 T cell can be measured by any means known in the art or described herein. For example, methylation can be determined by methylation-specific PCR, whole genome bisulfite sequencing, locus specific bisulfite sequencing, Ingenuity Pathway Analysis (IPA), the HELP assay and other methods using methylation-sensitive restriction endonucleases, ChIP-on-chip assays, restriction landmark genomic scanning, COBRA, Ms-SNuPE, methylated DNA immunoprecipitation (MeDip), pyrosequencing of bisulfite treated DNA, molecular break light assay for DNA adenine methyltransferase activity, methyl sensitive Southern blotting, methyl CpG binding proteins, mass spectrometry, HPLC, and reduced representation bisulfite sequencing. In some embodiments methylation is detected at specific sites of DNA methylation using pyrosequencing after bisulfite treatment and optionally after amplification of the methylation sites. Pyrosequencing technology is a method of sequencing-by-synthesis in real time. In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray.

In specific embodiments, detection of DNA methylation can be performed by first converting the DNA to be analyzed so that the unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. For example, isolated DNA can be treated with sodium bisulfite (NaHSO3) which converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. It is general knowledge that the resulting uracil has the base pairing behavior of thymine, which differs from cytosine base pairing behavior. To that end, uracil is recognized as a thymine by DNA polymerase. Therefore after PCR or sequencing, the resultant product contains cytosine only at the position where 5-methylcytosine occurs in the starting template DNA. This makes the discrimination between unmethylated and methylated cytosine possible.

The methylation status of CpG sites in test and controls samples may be compared by calculating the proportion of discordant reads, calculating variance, or calculating information entropy identifying differentially methylated regions, by quantifying methylation difference, or by gene-set analysis (i.e., pathway analysis), preferably by calculating the proportion of discordant reads, calculating variance, or calculating information entropy. Optionally, information entropy is calculated by adapting Shannon entropy. In some embodiments, gene-set analysis is performed by tools such as DAVID, GoSeq or GSEA. In some embodiments, a proportion of discordant reads (PDR) is calculated. Optionally, each region of neighboring CpG sites (e.g., within a sequencing read) is assigned a consistent status or an inconsistent status before calculating the proportion of discordant reads, variance, epipolymorphism or information entropy. There may be multiple inconsistent statuses, each representing a distinct methylation pattern or class of similar methylation patterns.

The CpG site identified for methylation analysis can be in a genomic feature selected from a CpG island, a CpG shore, a CpG shelf, a promoter, an enhancer, an exon, an intron, a gene body, a stem cell associated region, a short interspersed element (SINE), a long interspersed element (LINE), and a long terminal repeat (LTR). In specific embodiments, the CpG site is in a CpG island, a transcription factor, or a promoter within a given genomic locus, such as an effector locus.

In some embodiments, T-cell activity can be predicted based on the methylation status of a specific genomic locus or combination of genomic loci, referred to herein as a memory cell methylation marker. Accordingly, a positive memory cell methylation marker refers to markers whose methylation status relative to the corresponding methylation status of the same marker of an appropriate control (e.g., naïve T cell) indicates increased T-cell activity compared to a naïve T cell. Likewise, a negative memory cell methylation marker refers to markers whose methylation status relative to the corresponding methylation status of the same marker of an appropriate control (e.g., naïve T cell) indicates equal or decreased T-cell activity compared to a naïve T cell. As used herein, an effector profile or effector-associated epigenetic program, can refer to one or more memory cell methylation markers that identify a different subset of CD8 memory cells. In specific embodiments, the methylation status of a memory cell methylation marker termed a T_{scm} marker or T_{scm} locus indicates a T_{scm} differentiation state. As used herein, a T_{scm} effector profile or T_{scm} profile, can refer to one or more memory cell methylation markers that identify a T_{scm} differentiation state.

The methylation status of an individual marker can be measured at any location within the memory cell methylation marker locus (“marker locus” or “effector locus”). Thus, a memory cell methylation marker can refer to a CpG site within a marker locus or effector locus. As used herein a marker locus includes, but is not limited to, the genomic region beginning 2 kb upstream of the transcription start site and ending 2 kb downstream of the stop codon for each memory cell methylation marker gene. Likewise, an effector locus includes, but is not limited to, the genomic region beginning 2 kb upstream of the transcription start site and ending 2 kb downstream of the stop codon for each gene encoding an effector molecule. The marker locus or effector locus can include the region beginning 1 kb upstream of the transcription start site and ending 1 kb downstream of the stop codon, beginning 500 bp upstream of the transcription start site and ending 500 bp downstream of the stop codon, beginning 250 bp upstream of the transcription start site and ending 250 bp downstream of the stop codon, beginning 100 bp upstream of the transcription start site and ending 100 bp downstream of the stop codon, beginning 50 bp upstream of the transcription start site and ending 50 bp downstream of the stop codon, or beginning 10 bp upstream of the transcription start site and ending 10 bp downstream of the stop codon of the memory cell methylation marker gene or gene encoding an effector molecule, respectively. In specific embodiments, the methylation status of an individual memory cell methylation marker can be measured at a CpG site within the genomic locus.

In specific embodiments, demethylation of a CpG site at the CCR7 and/or CD62L locus indicates an increased capacity for T-cells to traffic to sites of antigen presentation. In some embodiments, methylation of a CpG site at the T-bet and/or Eomes locus indicates increased T-cell activity. In certain embodiments, demethylation of a CpG site at the Foxp1 locus indicates increased T-cell activity. In some embodiments the methylation status of a CpG site in a transcription factor coding sequence at the T-bet, Eomes, and/or Foxp1 locus indicates increased T-cell activity. In some embodiments, demethylation of a CpG site about 500 bp upstream of the transcription start site (TSS) of the IFNγ coding sequence indicates increased T-cell activity. In some embodiments, demethylation of a CpG site about 500 bp upstream of the TSS of the granzyme K (GzmK) coding sequence indicates increased T-cell activity. In some embodiments, demethylation of a CpG site about 10 bp downstream of the TSS of the granzyme B (GzmB) coding sequence indicates increased T-cell activity. In some embodiments, demethylation of a CpG site about 1 kb upstream of the TSS of the perforin 1 (Prf1) coding sequence indicates increased T-cell activity. In particular embodiments, the demethylation of a CpG site in the promoter sequence of the IFNγ, GzmK, GzmB, and/or Prf1 locus indicates increased T-cell activity. In particular embodiments, methylation status of a CpG site at an effector-associated locus can be used to predict T-cell activity. As used herein, an “effector associated locus” or “effector locus” includes the coding sequence of any genes encoding proteins that participate in the effector function of CD8 T cells. Examples of effector associated loci include but are not limited to, CD95, CD122, CCR7, CD62L, T-bet, Eomes, Myc, Tcf7, Foxp1, IFNγ, GzmK, GzmB, and/or Prf1. In particular embodiments, CD122 can be a homeostasis-associated locus, CCR7 and CD62L can be referred to as lymphoid homing loci, and Myc, Tcf7, Tbet, and Eomes can be referred to as memory differentiation associated transcription factors. In particular embodiments, T_{scm} cells are positive for CD95, CD122, CCR7, and CD62L markers. In some embodiments a T_{scm} epigenetic marker is a located at the T-bet, Eomes, Myc, Tcf7, Foxp1, IFNγ, GzmK, GzmB, and/or Prf1 locus.

In particular embodiments, methylation status of a CpG site at a T_{scm} locus (i.e., T_{scm} marker or T_{scm} epigenetic marker) can be used to predict a T_{scm} cell state or population of T_{scm} cells. In some embodiments, an effector-associated epigenetic program comprises demethylation of one or more of IFNγ, Perforin (Prf1), GzmB, and GzmK effector loci compared to the methylation status of the same effector loci in naïve CD8 T cells. In specific embodiments, demethylation of a CpG site at the CCR7 and/or CD62L locus is a T_{scm} locus and indicates an increased capacity for T-cells to traffic to sites of antigen presentation. In some embodiments, methylation of a CpG site at the T-bet and/or Eomes locus is a T_{scm} locus and indicates increased effector function and/or proliferative capacity. In certain embodiments, demethylation of a CpG site at the Foxp1 locus is a T_{scm} locus and indicates increased effector function and/or proliferative capacity. In some embodiments the methylation status of a CpG site in a transcription factor coding sequence at the T-bet, Eomes, and/or Foxp1 locus is a T_{scm} locus and indicates increased effector function and/or proliferative capacity. In some embodiments, demethylation of a CpG site about 500 bp upstream of the transcription start site (TSS) of the IFNγ coding sequence is a T_{scm} locus and indicates increased effector function. In some embodiments, demethylation of a CpG site about 500 bp upstream of the

TSS of the granzyme K (GzmK) coding sequence is a T_{scm} locus and indicates increased effector function and/or proliferative capacity. In some embodiments, demethylation of a CpG site about 10 bp downstream of the TSS of the granzyme B (GzmB) coding sequence is a T_{scm} locus and indicates increased effector function and/or proliferative capacity. In some embodiments, demethylation of a CpG site about 1 kb upstream of the TSS of the perforin 1 (Prf1) coding sequence is a T_{scm} locus and indicates increased effector function and/or proliferative capacity. In particular embodiments, the demethylation of a CpG site in the promoter sequence of the IFNγ, GzmK, GzmB, and/or Prf1 locus are a T_{scm} loci and indicates increased effector function and/or proliferative capacity. As used herein a Tscm profile or Tscm epigenetic profile refers to one or more than one epigenetic markers that can differentiate a Tscm cell from other memory cells, including but not limited to: CD95, CD122, CCR7, and CD62L, and markers at the T-bet, Eomes, Myc, Tcf7, Foxp1, IFNγ, GzmK, GzmB, and/or Prf1 loci.

Populations of T cells incubated with a signal 3 cytokine can be selected based on the methylation status of an individual locus or a combination of loci of a sample of T cells taken from the population. In some embodiments, T cell populations are selected based on measurement of the methylation status of any marker locus listed herein. In specific embodiments, selected T-cell populations comprise at least 30%, 40%, 50%, 55%, 60%, 65%, 70%, 75%, 80%, 90%, 95%, or more CD8 T cells having at least one positive T_{scm} marker, T_{scm} profile, or memory cell methylation marker. Accordingly, CD8 T cell populations selected by the methods disclosed herein comprising at least 30%, 40%, 50%, 55%, 60%, 65%, 70%, 75%, 80%, 90%, 95%, or more CD8 T cells having at least one positive T_{scm} marker, T_{scm} profile, or memory cell methylation marker. In some embodiments, the positive memory cell marker is associated with T_{scm} cells. Thus, CD8 T cells incubated with a signal 3 cytokine can be selected based on the methylation status of an individual locus (i.e., methylation marker) or a combination of loci (i.e., methylation profile) that is associated with T_{scm} cells.

Accordingly provided herein are methods of preparing a papulation of CD8 T_{scm} cells exhibiting at least one T_{scm} marker or combination of T_{scm} markers, comprising the steps of: culturing naïve CD8 T cells obtained from a mammal in vitro in the presence of IL-12 and, optionally, a stimulating factor. In specific embodiments, the cells are then analyzed to identify T_{scm} cells having at least one T_{scm} marker. Following culturing with IL-12, the cells can be enriched for cells expressing a marker selected from among CD95, CD122, CD28, CD62L, CCR7, CD127 and CD27.

In certain embodiments, the present invention provides for a pharmaceutical composition comprising a CD8 T cell incubated with a signal 3 cytokine and selected by the method disclosed herein, or comprising a population of CD8 T cells comprising at least 30%, 40%, 50%, 55%, 60%, 65%, 70%, 75%, 80%, 90%, 95%, or more CD8 T cells having at least one positive T_{scm} marker, T_{scm} profile, or memory cell methylation marker following incubation with a signal 3 cytokine (e.g., IL-12), and activation, as disclosed herein. The CD8 T cell or T cell population can be suitably formulated and introduced into a subject or the environment of the cell by any means recognized for such delivery. In some embodiments, the pharmaceutical composition comprises a CAR T cell produced from a CD8 T cell selected based on the identification of at least one positive methylation marker disclosed herein following incubation with a signal 3 cytokine.

Such pharmaceutical compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. In some embodiment a synthetic carrier is used wherein the carrier does not exist in nature. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The pharmaceutical compositions can also be prepared in the form of suppositories (e.g., with conventional suppository bases such as cocoa butter and other glycerides) or retention enemas for rectal delivery.

In one embodiment, the CD8 T cell or population of CD8 T cells having been incubated with a signal 3 cytokine are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and animal studies with the T cells disclosed herein can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For a compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a T cell having been incubated with a signal 3 cytokine can include a single treatment or, preferably, can include a series of treatments.

The pharmaceutical compositions can be included in a kit, container, pack, or dispenser together with instructions for administration.

The present invention provides for both prophylactic and therapeutic methods of treating a subject at risk of (or susceptible to) a chronic disease or infection. “Treatment”, or “treating” as used herein, is defined as the application or administration of a therapeutic agent (e.g., a selected CD8 T cell) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., a selected T cell having been incubated with a signal 3 cytokine). Subjects at risk for the disease can be identified by, for example, one or a combination of diagnostic or prognostic assays as known in the art. Administration of a prophylactic agent can occur prior to the detection of, e.g., cancer in a subject, or the manifestation of symptoms characteristic of the disease or disorder, such that the disease or disorder is prevented or, alternatively, delayed in its progression.

Another aspect of the invention pertains to methods of treating subjects therapeutically, i.e., altering the onset of symptoms of the disease or disorder. These methods can be performed in vitro (e.g., by culturing the cell with the agent(s)) or, alternatively, in vivo (e.g., by administering the agent(s) to a subject). With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment according to that individual's drug response genotype, methylation profile, expression profile, biomarkers, etc. Pharmacogenomics allows a clinician or physician to target prophylactic or therapeutic treatments to patients who will most benefit from the treatment and to avoid treatment of patients who will experience toxic drug-related side effects.

Therapeutic agents can be tested in a selected animal model. For example, an epigenetic agent or immunomodulatory agent as described herein can be used in an animal model to determine the efficacy, toxicity, or side effects of treatment with said agent. Alternatively, an agent (e.g., a therapeutic agent) can be used in an animal model to determine the mechanism of action of such an agent. Accordingly, methods are provided herein for the treatment or prevention of a chronic infection or cancer by administering a CD8 T cell, or CAR T cell selected based on the methylation status of at least one memory cell methylation marker and having been incubated with a signal 3 cytokine.

### Embodiments

1. A method for modulating the activity of at least one CD8 T cell obtained from a mammal, the method comprising:
incubating the at least one CD8 T cell obtained from a mammal in the presence of a signal 3 cytokine,
wherein the at least one CD8 T cell incubated in the presence of a signal 3 cytokine exhibits an enhanced effector potential compared to the effector potential of a control CD8 T cell.

2. The method of embodiment 1, wherein the CD8 T cell is a CD8 memory T cell.

3. The method of embodiment 1 or 2, wherein the CD8 T cell exhibits a memory (T_{scm}) cell phenotype following incubation with the signal 3 cytokine.

4. The method of embodiment 3, wherein said T_{scm} cell expresses the CD95 and CD122 markers.

5. The method of any one of embodiments 1-4, wherein the incubating occurs in vitro or ex vivo.

6. The method of any one of embodiments 1-3, wherein incubation of the CD8 T cell in the presence of a signal 3 cytokine establishes an effector-associated epigenetic program.

7. The method of embodiment 6, wherein the effector-associated epigenetic program comprises demethylation of one or more of IFNγ, Perforin (Prf1), GzmB, and GzmK effector loci compared to the methylation status of the same effector loci in naïve CD8 T cells.

8. The method of embodiment 6 or 7, wherein the effector-associated epigenetic program comprises demethylation of the IFNγ locus compared to the methylation status of the IFNγ locus in naïve CD8 T cells.

9. The method of any one of embodiments 1-8, wherein the signal 3 cytokine is a type I interferon or IL-12.

10. The method of any one of embodiments 1-8, wherein the signal 3 cytokine is IL-12.

11. The method of any one of embodiments 1-10, wherein the CD8 T cell exhibits an enhanced effector response upon activation of the CD8 T cell.

12. The method of embodiment 11, wherein activation of the CD8 T cell comprises incubation with an anti-CD3 and/or anti-CD28 antibody.

13. The method of any one of embodiments 1-12, wherein the enhanced effector potential comprises an increase in cytokine production, increase in the formation of intracellular granules, increase in the loading of granules with effector agents, and/or an increase in the transport and exocytosis of effector agents.

14. The method of embodiment 13, wherein the effector agents are granzymes, perforins, and/or granulysins.

15. The method of any one of embodiments 1-14, further comprising introducing a heterologous antigen receptor into the at least one at least one CD8 T cell incubated in the presence of a signal 3 cytokine.

16. The method of embodiment 15, wherein the antigen receptor comprises a T cell receptor (TCR) or a functional non-TCR antigen receptor.

17. The method of embodiment 15 or 16, wherein the heterologous antigen receptor is a chimeric antigen receptor (CAR).

18. The method of embodiment 17, wherein the CAR comprises an extracellular antigen-recognition domain and an intracellular signaling domain comprising an ITAM-containing sequence and an intracellular signaling domain of a T cell costimulatory molecule.

19. The method of any one of embodiments 1-18, wherein the mammal is a human.

20. The method of embodiment 19, wherein the human has cancer or is at risk of developing cancer.

21. The method of embodiment 20, wherein said cancer is a lymphoma, a leukemia, non-small cell lung carcinoma (NSCLC), head and neck cancer, skin cancer, melanoma, or squamous cell carcinoma (SCC).

22. The method of any one of embodiments 19-21, wherein the CD8 T cell is administered to a subject.

23. The method of embodiment 22, wherein the human from which the CD8 T cell is obtained is the subject.

24. The method of embodiment 22, wherein the human from which the CD8 T cell is obtained is different from the subject.

25. The method of any one of embodiments 22-24, further comprising administering an ICB therapy.

26. A method for selecting a subset of CD8 T cells comprising
incubating the at least one CD8 T cell obtained from a mammal in the presence of a signal 3 cytokine;
measuring the methylation profile of at least one CD 8 T cell; and
separating a subset of CD8 T cells comprising at least one positive memory cell methylation marker.

27. The method of embodiment 26, wherein said positive memory cell methylation marker comprises an unmethylated T_{scm} locus.

28. The method of embodiment 26, wherein said positive memory cell methylation marker comprises an unmethylated memory cell methylation marker.

29. The method of any one of embodiments 26-28, wherein said memory cell methylation marker is located at the transcription factor loci for Tcf7, Myc, T-bet, eomesodermin (Eomes), and/or Foxp1.

30. The method of any one of embodiments 26-28, wherein said memory cell methylation marker is located in at least one CpG site in the CCR7 and/or CD62L loci.

31. The method of any one of embodiments 26-28, wherein said memory cell methylation marker is located within 1 kb of the transcription start site of a nucleic acid sequence encoding IFNγ, granzyme K, GzmB, or Prf1.

32. The method of any one of embodiments 26-31, wherein said method further comprises activating the at least one CD8 T cell following incubation in the presence of a signal 3 cytokine.

33. A population of CD8 T cells selected by the method of any one of embodiments 26-32.

34. A population of CD8 T cells comprising at least 60% CD8 T cells having an enhanced effector response when compared to a control CD8 T cell.

35. The population of CD8 T cells of embodiment 34, wherein the CD8 T cells are naïve CD8 T cells prior to incubation in the presence of a signal 3 cytokine.

36. The population of CD8 T cells of embodiment 34 or 35, wherein the CD8 T cells are stem cell memory (T_{scm}) cells.

37. The population of CD8 T cells of any one of embodiments 33-36, wherein the at least 60% CD8 T cells having an enhanced effector response further comprise at least one positive T_{scm} marker.

38. The population of CD8 T cells of any one of embodiments 33-37, wherein at least 50% of the CD8 T cells further comprise a chimeric antigen receptor.

39. A pharmaceutical composition comprising said population of CD8 T cells of any one of embodiments 33-38.

40. A method of treating a chronic infection or cancer in a subject, said method comprising:
administering at least one CD8 T cell having enhanced effector potential compared to the effector potential of a control CD8 T cell, wherein the CD8 T cell was incubated in the presence of a signal 3 cytokine.

41. The method of embodiment 40, wherein the CD8 T cell is a CD8 memory T cell.

42. The method of embodiment 40 or 41, wherein the CD8 T cell is a stem cell memory (T_{scm}) cell.

43. The method of any one of embodiments 40-42, wherein the CD8 T cell exhibits at least one positive T_{scm} marker.

44. The method of any one of embodiments 40-43, wherein the signal 3 cytokine is a type I interferon or IL-12.

45. The method of any one of embodiments 40-44, wherein the CD8 T cell exhibits an enhanced effector response upon activation of the CD8 T cell.

46. The method of any one of embodiments 40-45, further comprising administering an ICB therapy.

47. Use of a signal 3 cytokine for enhancing the effector potential of a CD8 T cell comprising incubating a CD8 T cell in the presence of said signal 3 cytokine.

48. Use of a CD8 T cell having enhanced effector potential in the treatment of a chronic infection or cancer in a subject, wherein said CD8 T cell was incubated in the presence of a signal 3 cytokine.

49. Use of a CD8 T cell having enhanced effector potential in the manufacture of a medicament for the treatment of a chronic infection or cancer in a subject, wherein said CD8 T cell was incubated in the presence of a signal 3 cytokine.

### Experimental

### Example 1

IL-12 establishes stable demethylation programs of the IFNγ locus during in vitro naïve human CD8 T-cell differentiation

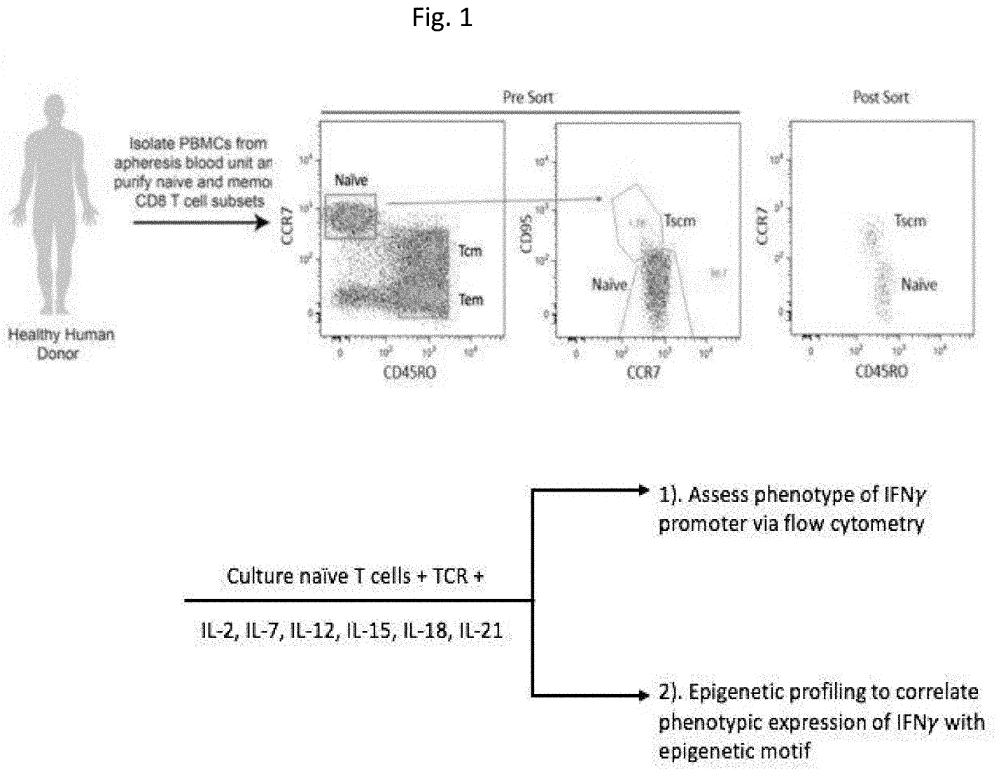

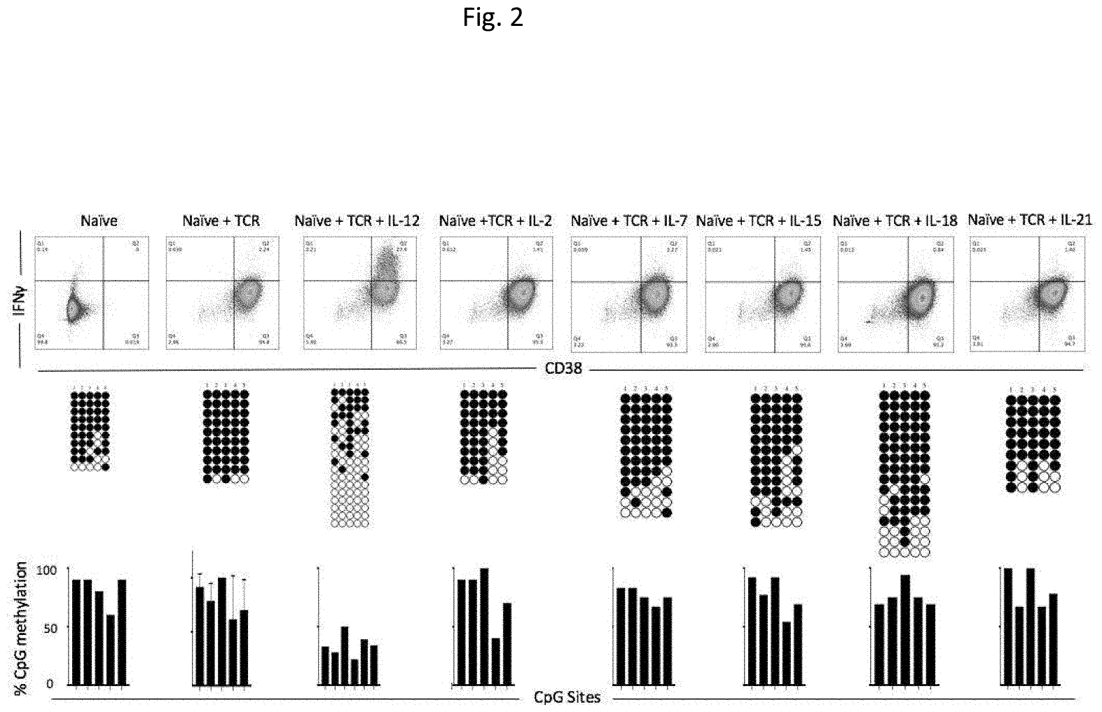

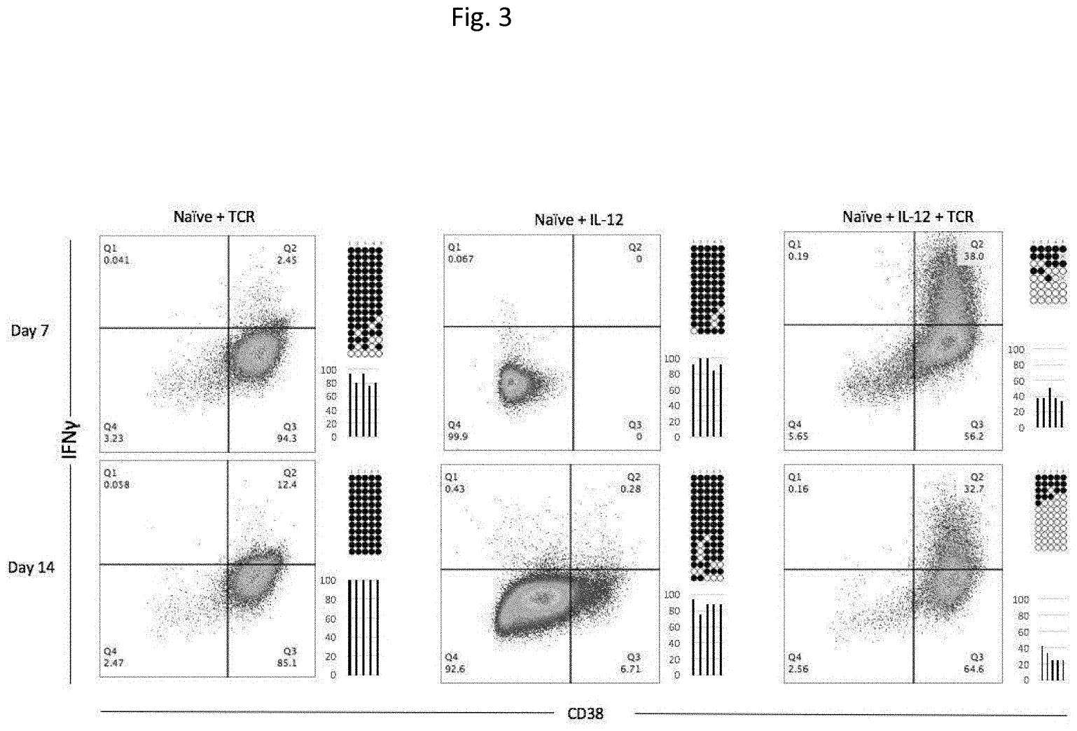

FIGS. 1, 2, and 3 confirm that incubation with IL-12 establishes stable demethylation programs of the IFNγ locus indicative of Tscm cells during in vitro naïve human CD8 T-cell differentiation.

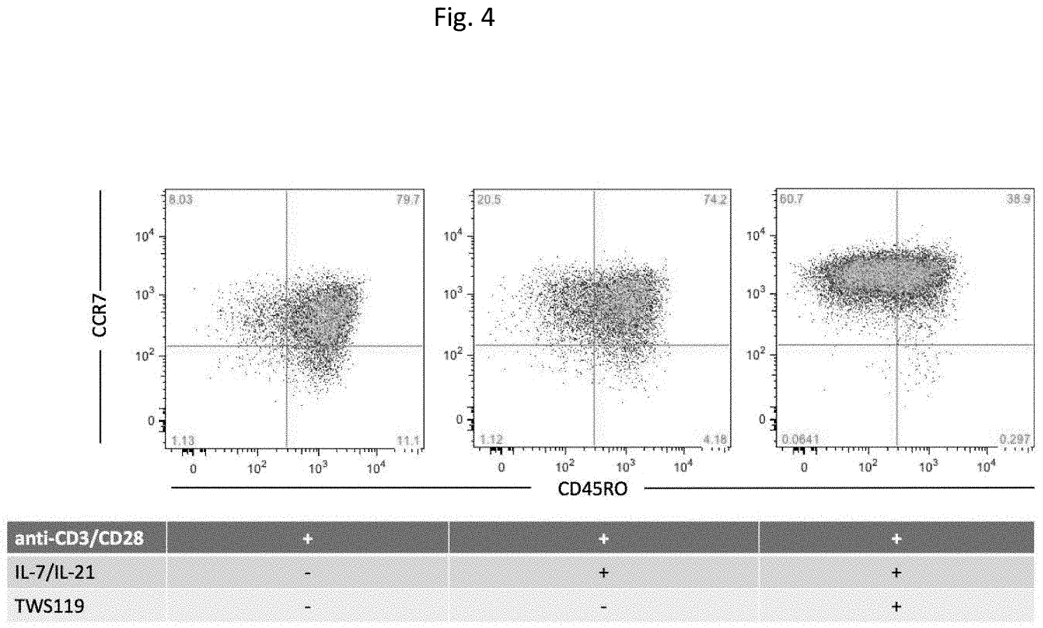

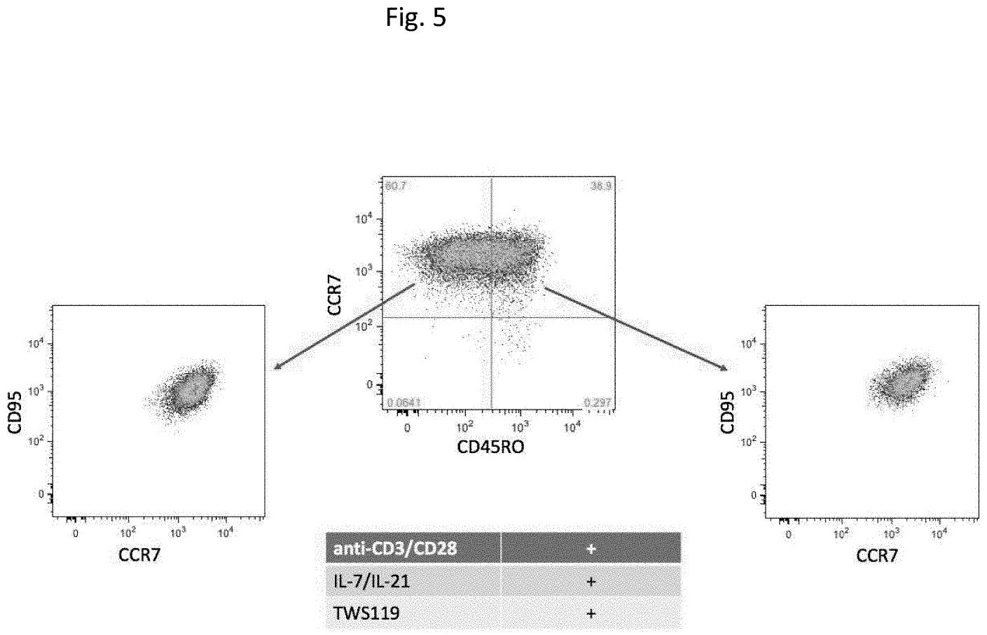

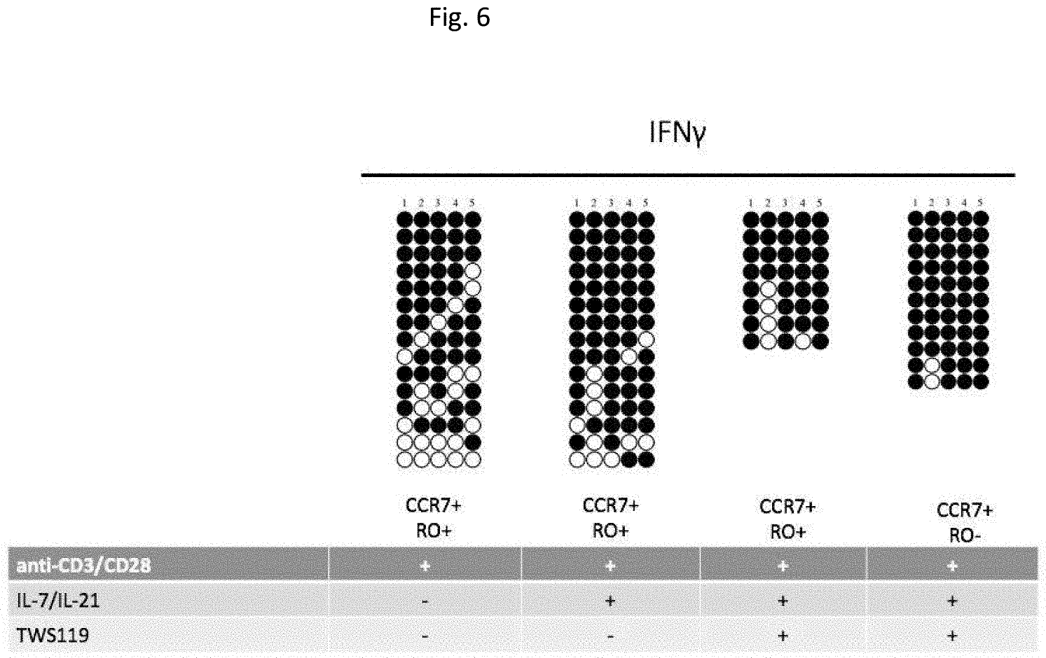

FIGS. 4, 5, and 6 demonstrate that established cell culture conditions for generating human memory CD8 T-cell phenotypes do not promote effector programs.

### Example 2

Coinfection induces bystander epigenetic demethylation of the IFNγ locus during the priming phase

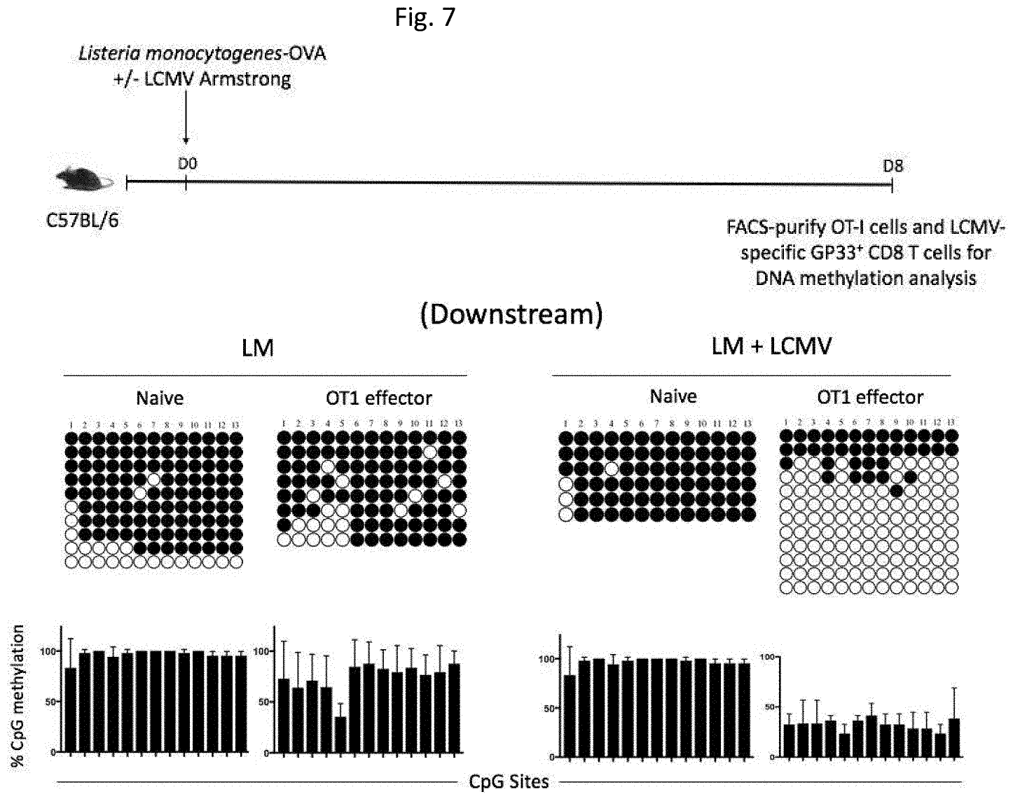

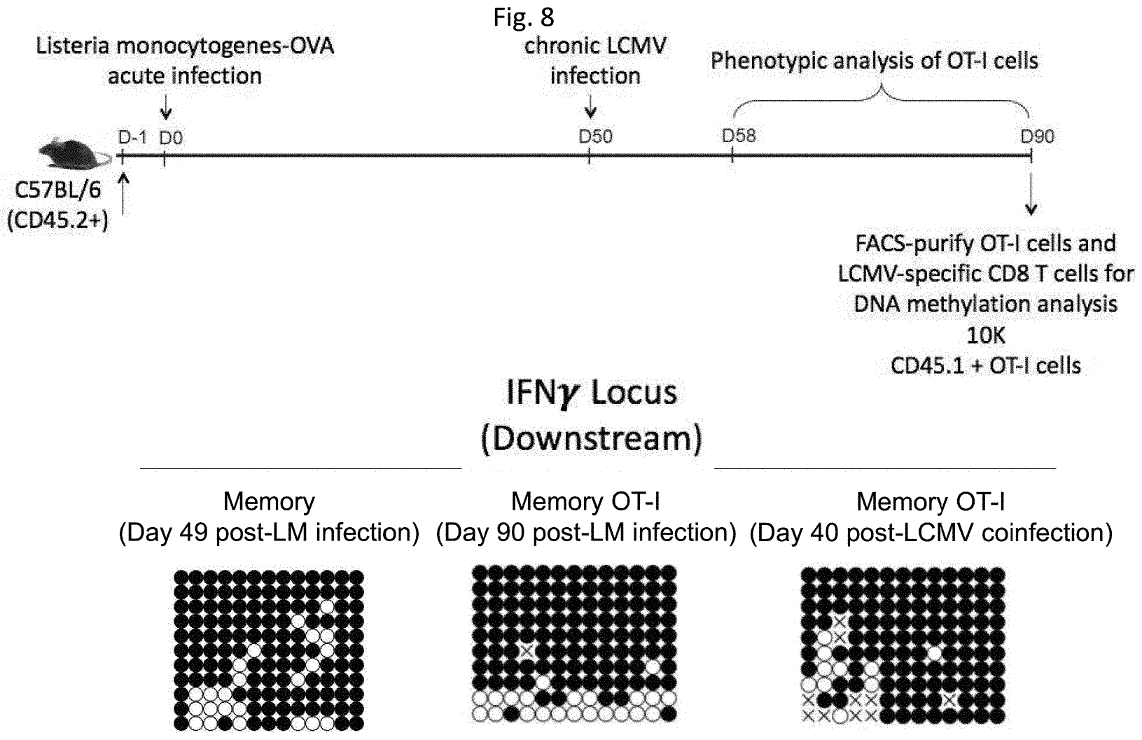

In vivo experiments with coinfection of C57BL/6 mice with Listeria monocytogenes and LCMV Armstrong induce an inflammatory environment that results in epigenetic poising of the downstream IFNγ locus, as presented in FIGS. 7 and 8. Demethylation of the IFNγ locus likely results from induction of cytokines in response to the infection. On account of epigenetic profiles being preserved during in vivo homeostasis, cytokines can be used to engineer a desired T cell population at the epigenetic level that maintains effector functions and proliferative capacity.

### Example 3

Human memory CD8 T-cell effector-potential is epigenetically preserved during in vivo homeostasis.

Immunological memory is a cardinal feature of adaptive immunity that provides a significant survival advantage by protecting individuals from previously encountered pathogens. Memory CD8 T cells, in particular, have the potential to provide life-long protection against pathogens containing their cognate epitope and are currently being exploited for strategies to protect against various intracellular pathogens and cancer cells. To achieve such long-lived protection, an adequate number of functionally competent memory CD8 T cells must be sustained in the absence of antigen through cytokine-driven homeostatic proliferation. Homeostasis of memory CD8 T cells is predominantly mediated by IL-7 and IL-15-induced expression of pro-survival genes and cell cycle regulators respectively. However, the cell-intrinsic mechanism(s) underlying stable maintenance of acquired effector functions during homeostatic proliferation remains largely unknown. Mounting evidence suggests that DNA-methylation programming is a primary mediator for preserving transcriptionally repressive and permissive chromatin states in cells that have undergone several rounds of division. Therefore, to gain insight into the potential epigenetic basis for maintenance of acquired properties among human memory CD8 T cells whole-genome bisulfite sequencing (WGBS) of sorted primary human naïve, shorter-lived Tem, and long-lived Tcm and Tscm CD8 T cells from healthy donors was performed.

Our initial assessment of genome-wide DNA methylation levels revealed that the overall number of methylated CpGs was inversely correlated with the established differentiation state of these cells: naïve>Tscm>Tcm>Tem. Moreover, the progressive decline in DNA methylation occurred across all autosomal chromosomes, indicating that effector and memory T cell differentiation is coupled to broad changes in DNA methylation. The higher level of methylation among long-lived memory CD8 T cells prompted us to further assess the relationship between naïve and memory CD8 T cell methylation profiles. An unsupervised principal component analysis (PCA) was performed on the methylation status of all CpG sites across the genome. Clustering was also observed among the naïve replicates as well as among T_{scm} replicates; importantly, the naïve and T_{scm} samples were found to be epigenetically distant. On the basis of the methylation status at 9,377,480 CpGs (CpG sites with >5× sequencing coverage for every sample), we generated a dendrogram of all replicate samples. Calculation of Euclidean distances between each population in the dendrogram indicated that despite the higher level of global DNA methylation, long-lived memory CD8 T cells (Tscm) have DNA methylation programs that are distinct from naïve cells.

To better define the DNA methylation programs that distinguish memory CD8 T cells from naïve cells we performed a pair-wise comparison of naïve versus memory cell WGBS datasets identifying differences in DNA methylation at individual CpG sites across the genome. This comparison allowed us to define the number, distribution, and nature of differentially methylated regions (DMRs) between the genomes of naïve and memory T cell subsets. We observed the greatest number of demethylated regions in Tₑₘ cells relative to naïve T cells, followed by T_{cm} cells, and then T_{scm} cells, further confirming our PCA results that the Tₑₘ memory subset are the most epigenetically distinct population from naïve CD8 T. Regardless of the methylated versus demethylated status, the majority of the DMRs were enriched in the 5′-distal regions (1-50 Kb) suggesting an association with transcriptional regulatory regions.

We next sought to identify DNA methylation programs coupled to the unique properties of the individual memory T cell subsets. Again a pair-wise comparison of the methylation status between each memory subset was performed and we detected 201980, 62240, and 9026 DMRs unique to Tₑₘ, T_{cm}, and T_{scm} CD8 T cells respectively. Among the DMRs that delineate the Tₑₘ, T_{cm}, and T_{scm} CD8 T cells were subset-associated DMRs at CpG sites in the CCR7 and CD62L (SELL) loci. Both CCR7 and CD62L DMRs were significantly methylated in CD8 Tₑₘ cells while these regions remained predominantly unmethylated in naïve, T_{cm} and T_{scm} CD8 T cells, consistent with the relative level of expression of these molecules in the different cell subsets. Similar to the lymphoid-homing molecules, we observed striking differences in methylation status at the transcription factor loci for T-bet and eomesodermin (Eomes), both of which have well-established roles in CD8 T-cell effector and memory differentiation. Consistent with the relative level of gene expression, all memory CD8 T cells were generally demethylated at regions downstream of the TSS of T-bet and Eomes, relative to that in naïve T cells. Notably, the Eomes locus contained a greater level of methylation in T_{scm} cells relative to the Tₑₘ cells at each of the DMRs.

In contrast to the memory subset-specific DNA methylation programs found at lymphoid homing molecules and transcription factors, demethylation DMRs at loci of classically defined effector molecules including IFNγ, Perforin, GzmB, and GzmK were observed in all memory T cell subsets compared to naïve cells. Of particular note was the striking level of demethylation at these loci in the long-lived T_{scm} CD8 T cells. To more broadly characterize DMRs linked to memory T cell longevity, we performed an ingenuity pathway analysis (IPA) of gene associated with T_{scm} DMRs. The IPA upstream regulator analysis identified STAT3 among the top potential regulators of the T_{scm} DMR gene list, further linking memory CD8 T cell development and the epigenetic poising of effector function in long-lived memory T cells.

Having determined that the loci of several effector molecules in long-lived memory CD8 T cells contain an epigenetic program suggestive of transcriptional permissivity, we next sought to determine if the effector-associated loci were poised for rapid gene expression in response to TCR stimulation. Naïve and memory CD8 T-cell subsets were purified and then cultured in the presence of anti-CD3/CD28 antibodies. mRNA was isolated longitudinally from the naïve and memory CD8 T cell subsets at 0, 4, and 12 hours following stimulation and the level of IFNγ, GzmB, and Prf1 transcription after TCR stimulation was determined. Our results revealed that GZMB and PRF1 transcription is rapidly induced in T_{cm} and T_{scm} cells upon TCR ligation, while Tₑₘ cells maintained a constitutively high level of expression following TCR activation. Interestingly, the level of IFNγ mRNA was high in all resting memory CD8 T cell subsets relative to naïve cells but was further upregulated upon stimulation of the memory subsets. Similar to the heightened kinetics for gene expression, TCR stimulation of the purified memory CD8 T-cell subsets also resulted in a rapid increase in the production of GzmB in T_{cm} and T_{scm} cells, relative to that in naïve T cells. These results provide further evidence that the epigenetic status for the IFNγ, PRF1, and GZMB genes in T_{cm} and T_{scm} cells is coupled to the poising of effector molecule expression.

To further assess the ability of memory CD8 T-cell subsets to maintain a “poised-for-expression” gene expression program during antigen-independent proliferation, we measured the expression of IFNγ following in an in vitro model of homeostatic cytokine-driven cell proliferation. Purified naïve and memory CD8 T cell subsets were labeled with the cell proliferation tracking dye CFSE, and then cultured in the presence of the homeostatic cytokines IL-7 and IL-15 for 7 days. Indeed, our results confirm prior reports of human memory CD8⁺ T-cell subsets having a hierarchical capacity to undergo cytokine driven homeostatic proliferation, with T_{scm} cells having the highest level of proliferation to both cytokines (naive<Tₑₘ<T_{cm}<T_{scm}, having undergone three or more cell divisions). We next measured the poised-recall response in cells that had undergone cytokine-driven proliferation by assessing the level of IFNγ protein in undivided and divided CD8 T cells after TCR stimulation. Quite strikingly, after 7 days in culture with IL-7 and IL-15, divided memory CD8 T cells retained the ability to express elevated levels of IFNγ protein after 4 hours

TCR stimulation. The results suggest that human memory CD8 T cells retain a gene expression program during IL-7/IL-15 mediated proliferation that allows the cells to remain poised to elicit a rapid effector response.

Our WGBS methylation analyses of primary T cells serves as a “snapshot” of the epigenetic state of long-lived memory CD8 T cells but fails to reveal whether or not the DNA-methylation programs are stable during homeostasis. Having validated that DNA methylation status of many of the DMRs identified from our WGBS analyses, including the DMRs identified in the IFNγ and Prf1 loci, we proceeded to use our newly designed loci-specific assays to determine whether the methylation status would remain unchanged during in vitro cytokine-driven homeostatic proliferation. Naïve, Tₑₘ, T_{cm}, and T_{scm} CD8 T cell subsets were FACS purified, labeled with CFSE, and then maintained in culture with IL-7 and IL-15 for 7 days. After 7 days, we then FACS purified the undivided and divided (≥3 rounds of cell division) fraction of cells and measured their DNA-methylation status. The IFNγ locus remained fully demethylated in all memory T-cell subsets that had undergone cell division, compared to naïve CD8 T cells. Moreover, naïve CD8 T cells that underwent more than three rounds of division retained a fully methylated IFNγ locus. These data demonstrate that cell division alone is not sufficient to demethylate the IFNγ locus in naïve cells; rather the process of demethylation is coupled to additional events/stages of memory T-cell differentiation.

Similar to the IFN□ locus, the demethylated status of CpGs within the Prf1 locus remained unchanged in dividing CD8 Tₑₘ cells. This region of the Prf1 locus was approximately 50% demethylated in resting CD8 T_{cm} and T_{scm} cells, which enabled us to test whether memory T cells undergo further demethylation through passive mechanisms (i.e., failure to propagate a methylation program during cell division). Remarkably, the 50% methylation status at the CpG sites in the T_{cm} and T_{scm} cells was faithfully propagated for more than three rounds of cell division, demonstrating that acquired epigenetic programs at effector-associated loci can persist during cytokine-drive homeostatic proliferation.

Antigen-independent phenotypic conversion of memory CD8 T cells occurs during in vivo and in vitro homeostatic proliferation but it remains openly debated whether this phenotypic conversion represents bone fide reprogramming of the cell's differentiation state. Indeed, culturing naïve, Tₑₘ, T_{cm}, and T_{scm} CD8 T cells with IL-7/IL-15 for 7 days results in a down-regulation of CCR7 expression in both T_{cm} and T_{scm} and a conversion to Tₑₘ-like cells. This observation promoted us to investigate the status of DNA methylation in CCR7 and CD62L DMRs under these conditions. We first confirmed that the CpG sites in the CCR7 and CD62L DMRs were fully demethylated in both naïve and T_{scm} cells and significantly methylated in Tₑₘ cells isolated from six independently sorted samples. These data further substantiate the link between CCR7 and CD62L expression and the methylation status of the DMRs. We next measured the methylation status of CCR7 and CD62L CpGs during cytokine-driven proliferation using the loci-specific assay. Naïve and memory CD8 T cell subsets were again cultured in the presence of IL-7 and IL-15 and the methylation assay was performed on purified undivided and divided populations. Similar to our findings with the IFNγ and Prf1 DMRs, the methylation status of the CCR7 and CD62L DMR CpGs in divided naïve CD8 T cells remained unchanged. However, we detected a significant increase in the methylation levels at the CCR7 DMR in divided T_{scm} cells. These results provide compelling evidence that cytokine-induced developmental changes among long-lived memory CD8 T cells are coupled to the cell's ability to undergo selective epigenetic reprogramming.

Collectively, the results from our in vitro homeostasis studies establish that DNA methylation programs associated with the heightened recall of effector functions are preserved over several rounds of cytokine-driven cell division, while programs coupled to homing and broadly used to delineate memory T cell subsets, can be modified. Although the effector-associated epigenetic programs exhibited remarkable stability under conditions of in vitro homeostasis, a lingering question is whether such stability occurs in vivo. One of the main challenges of studying in vivo human T cell homeostasis is the difficulty of tracking and re-isolating adoptively transferred T cells from the recipient due to their low frequency in circulation and the lack of congenic markers to distinguish donor versus recipient T cells. To overcome these challenges we took advantage of a novel T-cell depletion strategy utilized at our institution that selectively depletes CD45RA+ cells in haploidentical donor grafts for hematopoietic cell transplantation, thereby providing adoptive transfer of numerous donor memory cells at the time of transplantation. This infusion of polyclonal total Tcm and Tem memory T cells provides a unique opportunity to assess stability of epigenetic programs in human memory CD8 T cells during in vivo homeostatic proliferation.

Using the transplantation procedure we proceeded to assess the stability of DNA methylation programs in memory CD8 T cells that underwent antigen-independent expansion in vivo. Five blood samples from hematopoietic cell transplant recipients were selected for analyses based on the criteria of 100% donor chimerism among the reconstituted immune cells after infusion and no signs of immunological responses to infection. Donor T cells were phenotypically characterized prior to CD45RO enrichment for adoptive transfer and then characterized again ˜2 months after adoptive transfer and expansion in the patient. CD8 T cells isolated from the blood of recipients were strikingly void of cells exhibiting a naïve phenotype indicating that enrichment prior to infusion indeed excluded CD45RO-cells. The expanded CD8 T cells predominantly exhibited a Tₑₘ phenotype, despite the transfer of both T_{cm} and Tₑₘ memory CD8 T cell, and also expressed significantly higher levels of Ki67 indicating that they had recently proliferated. Notably, memory CD8 T cells isolated from the recipients had only a modest increase in the level of PD-1 expression, further supporting the conclusion that the majority of memory T cells in these patients had not recently encountered pathogen-associated antigens.

Having established that the majority of T cells isolated from the PBMCs of recipients retained a memory phenotype and originated from the donor (chimerism was 100% based on VNTR), we next sought to determine the DNA methylation status of effector and homing-associated DMRs in these cells. Loci-specific DNA methylation profiling of the IFNγ and Prf1

DMRs in purified donor Tem CD8 T cells (pre-transfer) and Tem-phenotyped cells isolated from the recipients confirmed that the promoters of these effector-associated genes remained demethylated during in vivo memory T cell reconstitution of the recipients. These data unambiguously establish that memory T cells can maintain a transcriptionally permissive epigenetic program at effector-associated loci during in vivo antigen-independent proliferation. Additionally, the CCR7 and CD62L DMRs were heavily methylated in the recipient memory T cells compared to the input donor memory T cells. Therefore, despite the donor infusion containing both T_{cm} and Tₑₘ CD8 T cells, the recipient was found to have primarily Tₑₘ CD8 T cells. It is quite possible that the absence of Tcm-like CD8 T cells from the circulation of the recipients' samples was due to selective death of the transferred T_{cm} or selective homing to the lymphoid tissue. Yet, a more exciting possibility is that these data represent in vivo evidence of memory CD8 T cell subset inter-conversion. Such conversion of Tem CD8 T cells into cells with a Tem phenotype is consistent with our in vitro results showing that gamma chain cytokines promote the conversion of long-lived memory CD8 T cells into Tem memory CD8 T cells.

Over the lifetime of an organism, memory T cell homeostasis ensures protection against pathogens that the host was previously exposed to and is achieved in part, by a fine balance between the death and proliferation of those cells. This balance is largely orchestrated by the common cytokines IL-7, which is essential for cell survival, and IL-15, which promotes cell cycling. Our study establishes that in vivo preservation of effector potential during cytokine-mediated homeostasis of memory CD8 T cells is coupled to the ability of the cell to transcribe acquired DNA methylation programs to newly generated daughter cells. Moreover, these results reveal that stabilization of epigenetic programming occurs in a loci-specific manner, providing new insight into the mechanisms regulating memory T cell subset inter-conversion. Broadly these data highlight epigenetic programming as a mechanism memory T cells use to strike a balance between remaining adaptive to their current and future environment while also retaining a history of past events.

Isolation of human CD8 T cells from healthy donor blood: This study was conducted with approval from the Institutional Review Board of St. Jude Children's Research Hospital. Human peripheral blood mononuclear cells (PBMCs) were collected through the St. Jude Blood Bank, and samples for WGBS were collected under IRB protocol XPD15-086. PBMCs were purified from platelet apheresis blood unit by density gradient. Briefly, blood was diluted 1:2.5 using sterile Dulbecco's phosphate-buffered saline (Life Technologies). The diluted blood was then overlayed above Ficol-Paque PLUS (GE Healthcare) at a final dilution of 1:2.5 (ficoll:diluted blood). The gradient was centrifuged at 400×g with no brake for 20 minutes at room temperature. The PBMCs interphase layer was collected and washed with 2% fetal bovine serum (FBS)/1 mM EDTA PBS buffer and then centrifuged at 400×g for 5 minutes. Total CD8 T cells were enriched from PBMCs by using the EasySep™ human CD8 negative selection kit (EasySep™, STEMCELL Technologies). Donors and patients were enrolled on an IRB approved protocol (registered at ClinicalTrials.gov, Identifier: NCT01807611), and provided informed consent for collection of the blood samples used for the in vivo analyses. Donor chimerism was determined utilizing CLIA-certified VNTR analysis.

Isolation and flow cytometric analysis naïve and memory CD8 T-cell subsets: Following enrichment of CD8 T cells, naïve and memory CD8 T-cell subsets were sorted using the following markers as previously described (23, 3I). Naïve CD8 T cells were phenotyped as live CD8⁺, CCR7⁺, CD45RO⁻, CD45RA⁺, CD95⁻ cells. CD8 T_{cm} cells were phenotyped as live CD8⁺, CCR7⁻, CD45RO⁺ cells. T_{cm} cells were phenotyped as live, CD8⁺, CCR7⁺, CD45RO⁺ cells. T_{scm} cells were phenotyped as live CD8⁺, CCR7⁺, CD45RO⁻,CD95⁺ cells. Sorted cells were checked for purity (i.e., samples were considered pure if more than 90% of the cells had the desired phenotype). Granzyme B expression was measured using sorted naïve or memory CD8 T-cell subsets stimulated with Dynabeads human T-cell activator CD3/CD28 at a 1:1 ratio. After approximately 18 hours of incubation at 37° C. and 5% CO₂, cells were harvested for cell-surface staining followed by intracellular staining.

Genomic Methylation Analysis: DNA was extracted from the sorted cells by using a DNA-extraction kit (Qiagen) and then bisulfite treated using an EZ DNA methylation kit (Zymo Research), which converts all unmethylated cytosines to uracils, while protecting methylated cytosines from the deamination reaction. The bisulfite-modified DNA-sequencing library was generated using the EpiGnome™ kit (Epicentre) per the manufacturer's instructions. Bisulfite-modified DNA libraries were sequenced using an Illumina Hiseq. Sequencing data were aligned to the HG19 genome by using BSMAP software. Differential-methylation analysis of CpG methylation among the datasets was determined using a Bayesian hierarchical model to detect regional methylation differences with at least three CpG sites. To perform loci-specific methylation analysis, bisulfite-modified DNA was PCR amplified with locus-specific primers (Supplemental Table). The PCR amplicon was cloned into a pGEMT easy vector (Promega) and then transformed into XL10-Gold ultracompetent bacteria (Stratagene). Bacterial colonies were selected using a blue/white X-gal—selection system after overnight growth, and then the cloning vector was purified and the genomic insert was sequenced. Following bisulfite treatment, the methylated CpGs were detected as cytosines in the sequence, and unmethylated CpGs were detected as thymines in the sequence by using BISMA software.

In vitro homeostatic proliferation: Sorted naïve CD8 T cells or memory CD8 T-cell subsets were labeled with CFSE (Life Technologies) at a final concentration of 2 μM. CFSE-labeled cells were maintained in culture in RPMI containing 10% FBS, penicillin-streptomycin, and gentamycin. Cells were maintained in culture with IL-7/IL-15 at a final concentration of 25 ng/mL each. After 7 days of incubation at 37° C. and 5% CO₂, undivided and divided cells (third division and higher) were sorted. Sorted cells were checked for purity (>90%). To determine whether the effector-recall response was maintained, we stimulated naïve and memory CD8 T-cell subsets with anti-CD3/CD28 beads (1:1) ratio for 4.5 hours in the presence of Golgi Stop and Golgi Plug after a 7-day exposure to IL-7/IL-15 in culture and then examined the levels of IFNγ protein expression by intracellular staining. For GzmB, cells were stimulated for 18 hrs with anti-CD3/CD28 beads (1:1) ratio.

Quantitative Transcriptional Analysis: Total RNA was extracted from naïve and memory CD8⁺T-cell subsets by using RNeasy plus micro kit (Qiagen). RNA was reverse transcribed into cDNA by using Superscript III reverse transcriptase (Roche Applied Science). Real-time PCR was performed on a CFX96 Real-time System (BioRad). Relative quantities of mRNA were determined using the Syber Select Master Mix CFX (Roche Applied Biosciences). Primer sequences are provided in the Supplementary Materials. The levels of mRNA for each gene were normalized to that of β-actin, and the fold increase in signal over naïve CD8 T cells was determined.

We claim:

1. A method for modulating the activity of at least one CD8 T cell obtained from a mammal, the method comprising:
incubating the at least one CD8 T cell obtained from a mammal in the presence of a signal 3 cytokine,
wherein the at least one CD8 T cell incubated in the presence of a signal 3 cytokine exhibits an enhanced effector potential compared to the effector potential of a control CD8 T cell.

2. The method of claim 1, wherein the CD8 T cell is a CD8 memory T cell.

3. The method of claim 1 or 2, wherein the CD8 T cell exhibits a memory (Teem) cell phenotype following incubation with the signal 3 cytokine.

4. The method of claim 3, wherein said T_{scm} cell expresses the CD95 and CD122 markers.

5. The method of any one of claims 1-4, wherein the incubating occurs in vitro or ex vivo.

6. The method of any one of claims 1-3, wherein incubation of the CD8 T cell in the presence of a signal 3 cytokine establishes an effector-associated epigenetic program.

7. The method of claim 6, wherein the effector-associated epigenetic program comprises demethylation of one or more of IFNγ, Perforin (Prf1), GzmB, and GzmK effector loci compared to the methylation status of the same effector loci in naïve CD8 T cells.

8. The method of claim 6 or 7, wherein the effector-associated epigenetic program comprises demethylation of the IFNγ locus compared to the methylation status of the IFNγ locus in naïve CD8 T cells.

9. The method of any one of claims 1-8, wherein the signal 3 cytokine is a type I interferon or IL-12.

10. The method of any one of claims 1-8, wherein the signal 3 cytokine is IL-12.

11. The method of any one of claims 1-10, wherein the CD8 T cell exhibits an enhanced effector response upon activation of the CD8 T cell.

12. The method of claim 11, wherein activation of the CD8 T cell comprises incubation with an anti-CD3 and/or anti-CD28 antibody.

13. The method of any one of claims 1-12, wherein the enhanced effector potential comprises an increase in cytokine production, increase in the formation of intracellular granules, increase in the loading of granules with effector agents, and/or an increase in the transport and exocytosis of effector agents.

14. The method of claim 13, wherein the effector agents are granzymes, perforins, and/or granulysins.

15. The method of any one of claims 1-14, further comprising introducing a heterologous antigen receptor into the at least one at least one CD8 T cell incubated in the presence of a signal 3 cytokine.

16. The method of claim 15, wherein the antigen receptor comprises a T cell receptor (TCR) or a functional non-TCR antigen receptor.

17. The method of claim 15 or 16, wherein the heterologous antigen receptor is a chimeric antigen receptor (CAR).

18. The method of claim 17, wherein the CAR comprises an extracellular antigen-recognition domain and an intracellular signaling domain comprising an ITAM-containing sequence and an intracellular signaling domain of a T cell costimulatory molecule.

19. The method of any one of claims 1-18, wherein the mammal is a human.

20. The method of claim 19, wherein the human has cancer or is at risk of developing cancer.

21. The method of claim 20, wherein said cancer is a lymphoma, a leukemia, non-small cell lung carcinoma (NSCLC), head and neck cancer, skin cancer, melanoma, or squamous cell carcinoma (SCC).

22. The method of any one of claims 19-21, wherein the CD8 T cell is administered to a subject.

23. The method of claim 22, wherein the human from which the CD8 T cell is obtained is the subject.

24. The method of claim 22, wherein the human from which the CD8 T cell is obtained is different from the subject.

25. The method of any one of claims 22-24, further comprising administering an ICB therapy.

26. A method for selecting a subset of CD8 T cells comprising
incubating the at least one CD8 T cell obtained from a mammal in the presence of a signal 3 cytokine;
measuring the methylation profile of at least one CD 8 T cell; and
separating a subset of CD8 T cells comprising at least one positive memory cell methylation marker.

27. The method of claim 26, wherein said positive memory cell methylation marker comprises an unmethylated T_{scm} locus.

28. The method of claim 26, wherein said positive memory cell methylation marker comprises an unmethylated memory cell methylation marker.

29. The method of any one of claims 26-28, wherein said memory cell methylation marker is located at the transcription factor loci for Tcf7, Myc, T-bet, eomesodermin (Eomes), and/or Foxp1.

30. The method of any one of claims 26-28, wherein said memory cell methylation marker is located in at least one CpG site in the CCR7 and/or CD62L loci.

31. The method of any one of claims 26-28, wherein said memory cell methylation marker is located within 1 kb of the transcription start site of a nucleic acid sequence encoding IFNγ, granzyme K, GzmB, or Prf1.

33. A population of CD8 T cells selected by the method of any one of claims 26-32.

34. A population of CD8 T cells comprising at least 60% CD8 T cells having an enhanced effector response when compared to a control CD8 T cell.

35. The population of CD8 T cells of claim 34, wherein the CD8 T cells are naïve CD8 T cells prior to incubation in the presence of a signal 3 cytokine.

36. The population of CD8 T cells of claim 34 or 35, wherein the CD8 T cells are stem cell memory (T_{scm}) cells.

37. The population of CD8 T cells of any one of claims 33-36, wherein the at least 60% CD8 T cells having an enhanced effector response further comprise at least one positive T_{scm} marker.

38. The population of CD8 T cells of any one of claims 33-37, wherein at least 50% of the CD8 T cells further comprise a chimeric antigen receptor.

39. A pharmaceutical composition comprising said population of CD8 T cells of any one of claims 33-38.

40. A method of treating a chronic infection or cancer in a subject, said method comprising:
administering at least one CD8 T cell having enhanced effector potential compared to the effector potential of a control CD8 T cell, wherein the CD8 T cell was incubated in the presence of a signal 3 cytokine.

41. The method of claim 40, wherein the CD8 T cell is a CD8 memory T cell.

42. The method of claim 40 or 41, wherein the CD8 T cell is a stem cell memory (T_{scm}) cell.

43. The method of any one of claims 40-42, wherein the CD8 T cell exhibits at least one positive T_{scm} marker.

44. The method of any one of claims 40-43, wherein the signal 3 cytokine is a type I interferon or IL-12.

45. The method of any one of claims 40-44, wherein the CD8 T cell exhibits an enhanced effector response upon activation of the CD8 T cell.

46. The method of any one of claims 40-45, further comprising administering an ICB therapy.

47. Use of a signal 3 cytokine for enhancing the effector potential of a CD8 T cell comprising incubating a CD8 T cell in the presence of said signal 3 cytokine.

48. Use of a CD8 T cell having enhanced effector potential in the treatment of a chronic infection or cancer in a subject, wherein said CD8 T cell was incubated in the presence of a signal 3 cytokine.

49. Use of a CD8 T cell having enhanced effector potential in the manufacture of a medicament for the treatment of a chronic infection or cancer in a subject, wherein said CD8 T cell was incubated in the presence of a signal 3 cytokine.
